(19) United States
(12) Patent Application Publication
(10) Pub. No.: US 2024/0091191 A1
(43) Pub. Date: Mar. 21, 2024

(54) USES OF CORYLIN AND/OR NEOBAVAISOFLAVONE IN TREATING SYMPTOMS ASSOCIATED WITH SENESCENCE

(71) Applicant: Chang Gung University, Taoyuan City (TW)

(72) Inventors: Chin-Chuan CHEN, Taoyuan City (TW); Yann-Lii LEU, Taoyuan City (TW); Shu-Huei WANG, New Taipei City (TW); Tong-Hong WANG, Taoyuan City (TW); Shu-Fang CHENG, Taoyuan City (TW)

(73) Assignee: Chang Gung University, Taoyuan City (TW)

(21) Appl. No.: 17/902,795

(22) Filed: Sep. 2, 2022

(51) Int. Cl. A61K 31/352; A61P 21/00; A61P 39/00

(52) U.S. Cl. CPC A61K 31/352; A61P 21/00; A61P 39/00

(57) ABSTRACT

Disclosed herein is related to a method for treating a symptom associated with senescence in a subject by administering to the subject with an effective amount of corylin and/or neobavaisoflavone, wherein the symptom associated with senescence is loss of muscle strength, muscle weakness, loss of motor coordination, loss of balance, skin wrinkle, or a poor blood biochemical parameter.

### STATEMENT REGARDING PRIOR DISCLOSURES BY THE INVENTOR OR A JOINT INVENTOR UNDER 37 C.F.R. 1.77(B)(6)

Part of the subject matter of the invention described in the present application was published by the inventors, Chin-Chuan Chen, Yann-Lii Leu, Shu-Huei Wang, Tong-Hong Wang, and Shu-Fang Cheng, in an article titled “The flavonoid corylin exhibits lifespan extension properties in mouse.” The article was published on Mar. 9, 2022 in the web site of Nature Communications. The publication was made by and/or originated from five members of the inventive entity of the present invention, and the entirety of this article is incorporated herein by reference. A copy of the article is provided in a concurrently filed Information Disclosure Statement pursuant to the guidance of 78 Fed. Reg. 11076 (Feb. 14, 2013).”

### BACKGROUND OF THE INVENTION

### 1. Field of the Invention

The present disclosure in general relates to the field of biological pharmaceuticals. More particularly, the present disclosure relates to corylin or neobavaisoflavone and their uses in treating symptoms associated with senescence.

### 2. Description of Related Art

Aging is a functional decline that occurs in all species, which may be caused in part by virtue of accumulating senescent cells in tissues and organs, which in turn leads to deteriorating biological functions and possibly contributing to aging-associated pathology. The dysregulated molecular mechanisms of aging such as loss of DNA repair capacity, chromosome instability, telomere erosion, and the like, elicit cell senescence, and the senescent cells then produce a senescence-associated secretory phenotype (SASP) to further trigger the senescence process in the surrounding tissue. Thus, the senescent cells deteriorate organ function and trigger aging-related disease, even raising the risk of death. Despite the fact that cellular senescence has a major impact on an individual's health, and discovery of anti-aging drugs may benefit in promoting healthy aging and in treating aging-related diseases, nonetheless, not many anti-aging drugs have been developed to date; only a few compounds with anti-aging activities have been described.

On the other hand, in accordance with records in several pharmacopoeias of traditional Chinese medicine (TCM), such as the Compendium of Materia Medica, Qianjinyaofang, Shennong Materia Medica, and Huangdi Neijing, many TCMs possess anti-aging activities. However, the key compounds from these TCMs have not been identified, hence their potential anti-aging activities have not been validated. As advances have been made in technologies for compound purification and in establishment of an in vitro screening platform for anti-aging drugs (e.g., the mother enrichment program (MEP)), it is now able to uncover potential anti-aging drugs from these TCMs. As described herein, one of the foregoing TCMs, Psoralea corylifolia (P. corylifolia), is of great interest, for it (especially the n-hexane-soluble fraction of P. corylifolia) exerts great potential in anti-aging activities.

In view of the foregoing, there exist a need of identifying active substances with anti-aging properties from P. corylifolia, in which the active substances may serve as candidate compounds for developing medicaments suitable for ameliorating cellular senescence, and/or treating aging-related diseases.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a method for treating a symptom associated with senescence in a subject, especially for symptoms regarding muscles, balance ability, or skins, or symptoms resulted from anomaly of blood biochemistry; the present method is used for increasing muscle strength or motor coordination; reducing muscle weakness, loss of balance, or skin wrinkle; or improving a blood biochemical parameter in a subject, comprising administering to the subject an effective amount of corylin and/or neobavaisoflavone.

Exemplary improved blood biochemical parameter may be any one of lower level of fasting blood glucose, lower level of total cholesterol, lower level of low-density lipoprotein, equal or higher level of high-density lipoprotein, lower level of triglyceride, lower level of aspartate transaminase, or lower level of creatinine, as compared with no corylin and/or neobavaisoflavone administered.

According to some embodiments of the present disclosure, the corylin and neobavaisoflavone are respectively administered to the subject in the amount of about 1-10 mg/kg and about 1-10 mg/kg.

Preferably, the subject treatable by the present method is a mammal, for example, a human, a mouse, a rat, a guinea pig, a hamster, a monkey, a swine, a dog, a cat, a horse, a sheep, a goat, a cow, and a rabbit. According to one preferred embodiment of the present disclosure, the subject is a human.

Many of the attendant features and advantages of the present disclosure will becomes better understood with reference to the following detailed description considered in connection with the accompanying drawings.

### BRIEF DESCRIPTION OF THE DRAWINGS

These and other features, aspects and advantages of the present invention will become better understood with reference to the following description, appended claims and the accompanying drawings, where:

FIGS. 1A-1H depict the effect of corylin or neobavaisoflavone on cellular senescence in accordance with the embodiments of the present disclosure. FIG. 1A is the result for assessment of the population doubling level (PDL) of human umbilical vein endothelial cells (HUVECs) under the indicated treatments. FIG. 1B is the western blotting result depicting the expression of p21 in HUVECs at PDL5 or PDL9 with or without treatment of corylin. FIG. 1C is the scatter plot summarizing the result of FIG. 1B. FIG. 1D is the scatter plot result depicting the ratio (%) of senescence-associated (SA)-β-gal-positive HUVECs at PDL5 or PDL9 with or without treatment of corylin. FIGS. 1E-1F are the results depicting the effect of corylin or neobavaisoflavone on ultraviolet B (UVB)-induced DNA breakage (FIG. 1E) and on UVB-induced cell apoptosis (FIG. 1F). FIGS. 1G-1H illustrate the effect of corylin (FIG. 1G) or neobavaisoflavone (FIG. 1H) on the level of intracellular NAD⁺;

FIGS. 2A-2F depict the effect of corylin on the physical functions of the aged high-fat diet (HFD)-fed mice. FIGS. 2A-2C are the results of the survival rate (FIG. 2A), the body weight (FIG. 2B), and the food intake (FIG. 2C) of the HFD-fed mice with or without corylin. FIG. 2D is the result of the rearing behavior of the HFD-fed mice with or without corylin. FIGS. 2E-2F are the results of the latency to falling in the constant rotarod tests (FIG. 2E) or in the accelerating rotarod tests (FIG. 2F) of the HFD-fed mice with or without corylin; and

FIGS. 3A-3F illustrate the improving effect of corylin on blood biochemical parameters of the HFD-fed mice, in which the blood biochemical parameters are fasting blood glucose (FIG. 3A), total cholesterol (FIG. 3B), low-density lipoprotein (LDL) (FIG. 3C), triglyceride (TG) (FIG. 3D), aspartate transaminase (AST) (FIG. 3E), and creatinine (FIG. 3F).

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a,” “an,” and “the” include the plural reference unless the context clearly dictates otherwise. Also, as used herein and in the claims, the terms “at least one” and “one or more” have the same meaning and include one, two, three, or more. The practice of the present invention will employ, unless otherwise indicated, conventional techniques of biochemistry, molecular biology, and the like, which are within the skill of the art. Such techniques are explained fully in the literature.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “symptoms associated with senescence” refers to any clinical manifestations that are related to physical deterioration, diseases, or disorders associated with aging, e.g., at a particular age; such symptoms may be resulted from dysfunctions of muscles (e.g., reduced muscle strength or enhanced muscle weakness), balance ability (e.g., reduced motor coordination or loss of balance), or skins (e.g., increased skin wrinkles), or may be resulted from aberration of blood biochemistry (e.g., higher level of fasting blood glucose, total cholesterol, low-density lipoprotein, triglyceride, aspartate transaminase, or creatinine; or lower level of high-density lipoprotein). The symptoms associated with senescence as described herein may be at least in part due to cellular senescence, and may further evolve into senescence-associated diseases, such as hypertension, sarcopenia, arthritis (e.g., degenerative arthritis, rheumatoid arthritis, metabolic arthritis, infectious arthritis, or spondylitis ankylosans), osteoporosis, or a neurodegenerative disease (e.g., Alzheimer's disease, vascular dementia, dementia with Lewy bodies, frontotemporal lobar degeneration, Parkinson's disease, Huntington's Disease, amyotrophic lateral sclerosis, multiple sclerosis, spinal muscular atrophy), which may be treatable with the present method.

A “decreased” or “reduced” level or amount is typically a “statistically significant” level or amount, and may include, for example, a 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) relative to a control. An decreased or reduced level or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold decrease (including all integers and ranges in between) relative to a control. Other examples of comparisons and “statistically significant” levels or amounts are described herein. “Decrease,” as used herein, can refer to “inhibit,” “reduce,” “curb,” “abate,” “diminish,” “lessen,” or “lower.”

A “increased” or “enhanced” level or amount is typically a “statistically significant” level or amount, and may include, for example, a 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase (including all integers and ranges in between) relative to a control. An increased or enhanced level or amount may also include a 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1,000-fold, 10,000-fold, or greater than 10,000-fold increase (including all integers and ranges in between) relative to a control. Other examples of comparisons and “statistically significant” levels or amounts are described herein. “Increase,” as used herein, can refer to “agonize,” “enhance,” “inflate,” “escalate,” expand,” “augment,” “enlarge,” or “raise.”

The term “statistically significant” refers to the result was unlikely to have occurred by chance. Statistical significance may be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less. The term “significant” encompasses and includes the term “statistically significant.”

The term “subject” or “patient” refers to an animal including the human species that is treatable with the pharmaceutically compositions and/or the methods of the present disclosure. The term “subject” or “patient” intended to refer to both the male and female gender unless one gender is specifically indicated. Accordingly, the term “subject” or “patient” comprises any mammal which may benefit from administration of corylin and/or neobavaisoflavone. Examples of a “subject” or “patient” include, but are not limited to, a human, rat, mouse, guinea pig, monkey, pig, goat, cow, horse, dog, cat, bird and fowl. In an exemplary embodiment, the patient is a human.

An “aged” animal, as used herein, refers to an adult animal that displays at least one phenotype associated with normal aging. The exact age that an animal will be considered “aged” depends on the species and/or the strain of the animal, as well as the phenotype in question, and can readily be determined by those of skill in the art.

The terms “treatment” and “treating” as used herein may refer to a curative or palliative measure. In particular, the term “treating” as used herein refers to the application or administration of the present corylin and/or neobavaisoflavone or a pharmaceutical composition comprising the same to a subject, who has a symptom or a disease associated with senescence, or a disease or a disorder secondary to the symptom or the disease associated with senescence, with the purpose to partially or completely alleviate, ameliorate, relieve, delay onset of, inhibit progression of, reduce severity of, and/or reduce incidence of one or more symptoms or features associated with senescence.

The term “administered,” “administering” or “administration” are used interchangeably herein to refer either directly administering the present corylin and/or neobavaisoflavone, or the present pharmaceutical compositions comprising corylin and/or neobavaisoflavone.

The term “an effective amount” as used herein refers to an amount effective, at dosages, and for periods of time necessary, to achieve the desired therapeutically desired result with respect to the treatment of symptoms or diseases associated with senescence in a subject. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/kg). Alternatively, the effective amount can be expressed in the concentration of the active component (e.g., the present corylin and/or neobavaisoflavone), such as molar concentration, mass concentration, volume concentration, molality, mole fraction, mass fraction and mixing ratio. Persons having ordinary skills could calculate the human equivalent dose (HED) for the medicament (such as the present antibody) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The term “pharmaceutical composition” refers to a preparation which is in such form as to permit the biological activity of an active ingredient contained therein to be effective, and which contains no additional components which are unacceptably toxic to a subject to which the composition would be administered. Usually, the amount of active compounds (i.e., corylin and/or neobavaisoflavone) is present in the pharmaceutical composition at a level of about 0.01% to 99% by weight; preferably, at a level of at least 0.1% by weight; more preferably, at a level of at least 1% by weight; even more preferably, at a level of at least 5% by weight; yet even more preferably, at a level of at least 10% by weight; still yet even more preferably, at a level of at least 25% by weight, based on the total weight of the pharmaceutical composition. For the clinical use of the present invention, the present pharmaceutical composition is formulated into formulations suitable for the intended route of administration.

The phrase “pharmaceutically acceptable excipient” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each excipient must be “acceptable” in the sense of being compatible with the other ingredients of the formulation. The pharmaceutical formulation contains a compound of the invention in combination with one or more pharmaceutically acceptable ingredients. The excipient can be in the form of a solid, semi-solid or liquid diluent, cream or a capsule. These pharmaceutical preparations are a further object of the invention.

### II. Description of the Invention

The present disclosure is based, at least in part, on the discovery that corylin and neobavaisoflavone are independently capable of improving physical functions of human bodies, such as promoting muscle health and improving balance, enhancing skin resistance, strengthening circulatory health, etc., accordingly, corylin and/or neobavaisoflavone may enhance the vitality of an individual, and eventually may confer the individual with rejuvenized characteristics.

### 1. The Present Compositions

Accordingly, the first aspect of the present disclosure is to provide a composition for treating symptoms or diseases associated with senescence as described herein. The composition comprises corylin and/or neobavaisoflavone, and a carrier. Said corylin and said neobavaisoflavone respectively have the chemical structures of formula (I) and (II):

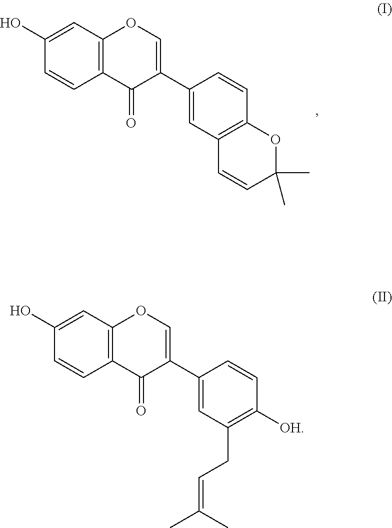 [SMILES: CC1(C)C=Cc2cc(-c3coc4cc(O)ccc4c3=O)ccc2O1 ; CC(C)=CCc1cc(-c2coc3cc(O)ccc3c2=O)ccc1O]

The corylin and/or the neobavaisoflavone comprised in the present composition may be obtained by extracting from natural herbs or by chemical synthesis. Said natural herbs include, but are not limited to, Psoralea corylifolia (babchi), Psoralea abbottii C. H. Stirt., Psoralea aculeata L., Psoralea affinis Eckl. & Zeyh., Psoralea alata (Thunb.) T. M. Salter, Psoralea angustifolia L'Hér., Psoralea aphylla L., Psoralea arborea Sims, Psoralea asarina (P. J. Bergius) T. M. Salter, Psoralea axillaris L.f., Psoralea azuroides C. H. Stirt., Psoralea brilliantissima C. H. Stirt., Muasya & A. Bello, Psoralea cataracta C. H. Stirt., Psoralea congesta C. H. Stirt. & Muasya, Psoralea diturnerae A. Bello, C. H. Stirt. & Muasya, Psoralea elegans C. H. Stirt., Psoralea ensifolia (Houtt.) Merr., Psoralea fascicularis DC. (syn. Psoralea tenuifolia Thunb., syn. Psoralea thunbergiana Eckl. & Zeyh.), Psoralea filifolia Eckl. & Zeyh., Psoralea fleta C. H. Stirt., Psoralea floccosa C. H. Stirt., Muasya & A. Bello, Psoralea forbesiae C. H. Stirt., A. Bello & Muasya, Psoralea gigantea Dludlu, Muasya & C. H. Stirt., Psoralea glabra E. Mey., Psoralea glaucescens Eckl. & Zeyh., Psoralea glaucina Harv., Psoralea gueinzii Harv., Psoralea imbricata (L.f.) T. M. Salter, Psoralea imminens C. H. Stirt., Psoralea implexa C. H. Stirt., Psoralea intonsa C. H. Stirt., Muasya & A. Bello, Psoralea ivumba C. H. Stirt., A. Bello & Muasya, Psoralea karooensis C. H. Stirt., Muasya & Vlok, Psoralea keetii Schönland ex H. M. L. Forbes, Psoralea kougaensis C. H. Stirt., Muasya & A. Bello, Psoralea laevigata L.f, Psoralea laxa T. M. Salter, Psoralea margaretiflora C. H. Stirt. & V. R. Clark, Psoralea monophylla (L.) C. H. Stirt., Psoralea montana A. Bello, C. H. Stirt. & Muasya, Psoralea muirii C. H. Stirt. & Muasya, Psoralea odoratissima Jacq., Psoralea oligophylla Eckl. & Zeyh., Psoralea oreophila Schltr., Psoralea peratica C. H. Stirt., Psoralea pinnata L., Psoralea plauta C. H. Stirt., Psoralea pullata C. H. Stirt., Psoralea ramulosa C. H. Stirt., Psoralea repens P. J. Bergius, Psoralea restioides Eckl. & Zeyh., Psoralea rhizotoma C. H. Stirt. & Muasya, Psoralea rigidula C. H. Stirt., Psoralea semota C. H. Stirt., Psoralea sordida C. H. Stirt. & Muasya, Psoralea speciosa Eckl. & Zeyh., Psoralea suaveolens C. H. Stirt., A. Bello & Muasya, Psoralea tenuifolia L., Psoralea tenuissima E. Mey., Psoralea triflora Thunb., Psoralea trullata C. H. Stirt., Psoralea usitata C. H. Stirt., Psoralea vanberkeliae C. H. Stirt., A. Bello & Muasya, Psoralea verrucosa Willd. ex Spreng., and Puerariae caulis (galman). Preferably, the corylin or the neobavaisoflavone may be extracted from Psoralea corylifolia (babchi), via any extraction methods known in the art. Preferably, the corylin and/or the neobavaisoflavone may be obtained by mixing any of the herbs described above with an extractant such as ethanol, n-hexane, ethyl acetate, and etc. for a sufficient period of time to produce a crude extract.

Additionally or alternatively, the crude extract may be further purified to give substantially pure corylin and neobavaisoflavone. Generally, the crude extract is purified by column chromatography (e.g., high performance liquid chromatography (HPLC), silica gel chromatography), thin-layer chromatography, and the like. In one working example, the crude extract is subjected to silica gel chromatography to give substantially pure corylin and neobavaisoflavone.

The purified compound is then identified by methods including, but are not limited to, chemical analysis (e.g., titration analysis, gravimetric analysis, or micro-spectroscopy), spectroscopic analysis (e.g., optical rotation spectroscopy, atomic spectroscopy, or molecular spectroscopy), mass spectrometry, nuclear magnetic resonance (NMR) proton spectroscopy, chromatography (e.g., column chromatography, ion-exchange chromatography, gel chromatography, affinity chromatography, HPLC, or thin-layer chromatography), infrared spectroscopy, or a combination thereof (e.g., liquid chromatography-mass spectrometry (LC-MS)). In one working example, the corylin and the neobavaisoflavone are identified by NMR proton spectroscopy.

The composition comprising corylin and/or neobavaisoflavone may be utilized in the form of a pharmaceutical (including a nutritional supplement), a cosmetic, or a health functional food according to its purposes.

According to some embodiments of the present disclosure, the composition is used as the pharmaceuticals (or the nutritional supplement), in such case, the pharmaceutical composition may further comprise suitable carriers, excipients, and diluents commonly used in the manufacture of pharmaceutical compositions. In addition, the pharmaceutical composition may be formulated and used in the form of oral dosage forms such as powders, granules, tablets, capsules, suspensions, emulsions, syrups, aerosols, external preparations, suppositories; and sterile injectable solutions according to a conventional method. Carriers, excipients, and diluents that may be included in the pharmaceutical composition comprise lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. In the case of formulation, it is prepared using excipients or diluents such as fillers, extenders, binders, wetting agents, disintegrating agents, and surfactants. Solid preparations for oral administration include tablets, pills, powders, granules, capsules, etc. These solid preparations include at least one excipient in the preparation, for example, starch, calcium carbonate, sucrose, lactose, gelatin, and the like. In addition, lubricants such as magnesium stearate or talc are used in addition to simple excipients. Liquid preparations for oral use include suspending agents, solutions, emulsions, syrups, etc. In addition to water and liquid paraffin, which are commonly used as simple diluents, various excipients, such as wetting agents, sweeteners, fragrances, and preservatives, may be included in the preparation. Formulations for parenteral administration include sterile aqueous solutions, non-aqueous solvents, suspensions, emulsions, lyophilized preparations, and suppositories. Non-aqueous solvents and suspensions may include propylene glycol, polyethylene glycol, vegetable oils such as olive oil, and injectable esters such as ethyl oleate. As a base for suppositories, witepsol, macrogol, Tween-61, cacao butter, laurin butter, and glycerogelatin may be used.

According to some embodiments of the present disclosure, the composition is used as the cosmetics, in such case, components commonly used in the cosmetic composition may be included in addition to the active ingredient, for example, a stabilizer, a solubilizing agent, a common auxiliary agent such as vitamins, pigments and fragrances, and a carrier. In addition, the cosmetic composition may be prepared in any formulations conventionally prepared in the art, such as solutions, suspensions, emulsions, pastes, gels, creams, lotions, powders, soaps, surfactant-containing cleansings, oils, powders. It may be formulated as a foundation, emulsion foundation, wax foundation, spray, etc., but is not limited thereto. In such cases when the formulation is a paste, cream or gel, animal oil, vegetable oil, wax, paraffin, starch, tragacanth, cellulose derivative, polyethylene glycol, silicone, bentonite, silica, talc, zinc oxide, etc. may be used as carrier components. In addition, in the case when the formulation is a powder or a spray, lactose, talc, silica, aluminum hydroxide, calcium silicate, or polyamide powder may be used as a carrier component, and especially in the case of a spray, additionally propellants such as chlorofluorohydrocarbon, propane/butane, and dimethyl ether may be used. In addition, when the formulation is a solution or an emulsion, a solvent, a solubilizing agent or an emulsifying agent is used as a carrier component, such as water, ethanol, isopropanol, ethyl carbonate, ethyl acetate, benzyl alcohol, benzyl benzoate, propylene glycol, fatty acid esters of 3-butyl glycol oil, glycerol aliphatic esters, polyethylene glycol, or sorbitan may be used. Also, when the formulation is a suspension, liquid diluents such as water, ethanol, and propylene glycol, ethoxylated isostearyl alcohol, suspensions such as polyoxyethylene sorbitol esters and polyoxyethylene sorbitan esters, microcrystalline cellulose, aluminum metahydroxide, bentonite, agar, trakant, and the like may be used as carrier components. In addition, when the above formulation is surfactant-containing cleansings, aliphatic alcohol sulfate, aliphatic alcohol ether sulfate, sulfosuccinic acid monoester, isethionate, imidazolinium derivatives, methyl taurate, sarcosinate, fatty acid amide ether sulfates, alkylamidobetaines, aliphatic alcohols, fatty acid glycerides, fatty acid diethanolamides, vegetable oils, lanolin derivatives, ethoxylated glycerol fatty acid esters, and the like may be used as the carrier component.

According to some embodiments of the present disclosure, the composition is utilized as the health functional food, ingredients commonly used in the health functional food may be included in addition to the active ingredient, for example, citric acid, oligosaccharide, taurine, fruit concentrate, etc. may be included when the composition is manufactured as a health functional beverage.

### 2. Uses of the Present Compositions

Another aspect of the present disclosure pertains to a method for increasing muscle strength or motor coordination; reducing muscle weakness, loss of balance, or skin wrinkle; or improving a blood biochemical parameter (i.e., the symptom or the disease associated with senescence) in a subject. The method comprises administering to the subject an effective amount of the composition described above, which comprises the corylin and/or neobavaisoflavone; and a carrier.

The pharmaceutical composition is administered to the subject in the amount of about 0.01 to 1,000 mg/kg body weight of the subject, such as 0.01, 0.02, 0.03, 0.04, 0.05, 0.06, 0.07, 0.08, 0.09, 0.1, 0.2, 0.3 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 550, 600, 650, 700, 750, 800, 850, 900, 950, or 1,000 mg/kg body weight of the subject; preferably, about 0.1 to 100 mg/kg body weight of the subject. According to some embodiments of the present disclosure, the present composition is administered to the subject in need thereof in the amount of about 1-10 mg/kg. In one working example, the present composition is administered to the subject in the amount of about 5 mg/kg. The dose can be administered in a single aliquot, or alternatively in more than one aliquot. The skilled artisan or clinical practitioner may adjust the dosage or regime in accordance with the physical condition of the patient or the severity of the diseases.

As would be appreciated, in the case when the subject is afflicted with a disease associated with senescence, the present method may be applied to the subject alone or in combination with an additional therapy (e.g., a surgery, a physical therapy, a physiotherapy, a massage, a phototherapy, or a thermotherapy) or an additional medicament that have some beneficial effects on treating of the symptom or the disease associated with senescence. Depending on the intended therapeutic purposes, the present method may be applied to the subject prior to, in conjunction with, or subsequent to the administration of the additional therapy or the additional medicament.

Said additional medicament may be an anti-hypertensive drug, such as aliskiren, amiloride, amlodipine, atenolol, bendroflumethiazide, bumetanide, candesartan, captopril, clonidine, diltiazem, doxazosin, furosemide, hydralazine, hydrochlorothiazide, labetalol, lisinopril, losartan, methyldopa, metoprolol, minoxidil, nifedipine, nimodipine, phenoxybenzamine, phentolamine, propranolol, ramipril, spironolactone, trimetaphan, valsartan, or verapamil; an anti-inflammatory drug, such as abatacept, acitretin, adalimumab, alefacept, anakinra, anthralin, apremilast, azathioprine, baricitinib, belimumab, benralizumab, betamethasone, bimekizumab, brazikumab, brodalumab, calcipotriene, calcipotriol, calcitriol, canakinumab, certolizumab, clobetasol, coal tar, colchicine, cyclosporine, dapsone, dithranol, dexamethasone, dupilumab, eculizumab, etanercept, fluocinolone, golimumab, guselkumab, hydroxychloroquine, hydrocortisone, infliximab, ixekizumab, lenercept, mepolizumab, methotrexate, methylprednisolone, mirikizumab, mycophenolate mofetil, omalizumab, prednisone, perakizumab, pimecrolimus, remtolumab, reslizumab, rilonacept, risankizumab, rituximab, sarilumab, secukinumab, sulfasalazine, tacrolimus, tazarotene, tildrakizumab, tocilizumab, tofacitinib, tretinoin, upadacitinib, ustekinumab, vedolizumab, or vunakizumab; an anti-osteoporotic drug, such as abaloparatide, alendronate, alfacalcidol, calcitonin, calcium, clodronate, denosumab, eldecalcitol, etidronate, ibandronate, minodronate, monofluorophosphate, pamidronate, parathyroid hormone, raloxifene, risedronate, romosozumab, sodium fluoride (NaF), teriparatide, vitamin D₃, or zoledronic acid; a drug for treating a neurodegenerative disease, such as acyclovir, aducanumab, amantadine, apomorphine, baclofen, carbidopa, dantrolene, donepezil, entacapone, foscarnet, galantamine, levodopa, memantine, penciclovir, pramipexole, rasagiline, riluzole, rivastigmine, ropinirole, selegiline, tacrine, tetrabenazine, tizanidine, or tolcapone.

It should be noted that during the term of the present treatment, different therapies or therapeutics may be administered to the subject at different doses, time intervals, via different routes. The doses and time intervals may vary with factors such as described above, and are dependent on the professional considerations of the practitioner; and the routes may be via oral, enteral, buccal, nasal, transdermal, transmucosal, intravenous, intraperitoneal, intraarterial, intracutaneous, subcutaneous, and intramuscular routes.

Basically, the subject treatable by the present method is a mammal; preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

1. Extraction and Isolation of Psoralea corylifolia

The dried fruits (5.4 kg) of Psoralea corylifolia were extracted with ethanol at room temperature for four times (11 liters each time), then with ethanol at 70° C. for 4 hours for five times (11 liters each time). The combined crude extracts (1.4 kg) were sequentially partitioned between H₂ O (842.38 g) and n-hexane (557.62 g). The n-hexane layer was separated by silica gel chromatography using mixtures of n-hexane and ethyl acetate, in which corylin and neobavaisoflavone were eluted when n-hexane:ethyl acetate=5:1.

### 2. Cell Culture

HUVECs were cultured in M199 medium supplemented with 4-(2-hydroxyethyl)-1-piperazineethanesulfonic acid (HEPES), endothelial cell growth supplement (ECGS; Millipore), heparin, NaHCO₃, L-glutamine, sodium pyruvate, and 20% fetal bovine serum (FBS; final concentration). HaCaT cells were cultured in DMEM supplemented with 10% FBS and 2 mM glutamine. The cells were grown in an atmosphere of 5% CO₂ at 37° C., and passaged to maintain under 90% confluency. The cells were propagated until senescence, and those in every passage were collected for further analysis, numbers of which were determined when sub-cultured. Population doublings (PDs) were estimated following the equation: PDL=3.32 (log(total viable cells at harvest/total viable cells at seeding).

### 3. Western Blot Analysis

The cells were harvested and lysed in RIPA lysis buffer containing 1% of Triton X-100 and a protease inhibitor cocktail, and total protein concentrations were measured with a protein assay kit. Proteins in the samples of the lysates with the same amounts of total protein were separated by sodium dodecyl sulfate polyacrylamide gel electrophoresis (SDS-PAGE) (10% polyacrylamide gels) and analyzed by immunoblotting. Primary antibodies against p21 and β-actin were applied to detect the corresponding proteins. Horseradish peroxidase (HRP)-conjugated secondary antibodies against mouse IgG were then incubated with the membranes, and proteins were visualized by chemiluminescence according to the manufacturer's instructions. Protein quantification was performed by software.

### 4. Senescence-Associated (SA)-β-gal Staining

HUVECs at 90% confluency were collected, washed, and fixed with 2% formaldehyde and 0.2% glutaraldehyde for 5 minutes. The cells were then subjected to staining by incubation with a staining solution (40 mmol/l citric acid, sodium phosphate, pH 6.0, 1 mg/ml 5-bromo-4-chloro-3-isolyl-β-D-galactoside (X-gal, Sigma), 5 mmol/l potassium ferrocyanide, 5 mmol/l potassium ferricyanide, 150 mmol/l NaCl, and 2 mmol/l MgCl₂) at 37° C. for 18 hours. The results of SA-β-gal-positive cells were observed by microscopy, and were counted for over 300 cells in at least three independent fields.

### 5. DNA Breakage Analysis

HaCaT cells were seeded into a 12-well plate (7×10⁴ cells/well), and incubated with 100 μM corylin or 100 μM neobavaisoflavone, or without drugs, for 24 hours, before subjected to UVB irradiation at 30 mJ/m². The irradiated cells were collected, washed, and mixed with 1% agarose gel solution 140 μl at 35° C., before being applied onto slides. The slides were placed at 4° C. for 1 hour for solidifying the gel, immersed in a lysis buffer for at least 1 hour and subsequently in an electrophoresis fluid for 40 minutes, and then subjected to gel-electrophoresis at 25 V for 20 minutes. The slides were immersed in a neutralizing solution for 5 minutes, mounted with 30 μl mounting medium with DAPI, and observed under a fluorescence microscope. The results were analyzed with the software OPEN COMET to quantify the breakage and the repair of the DNA in the cells.

### 6. Apoptosis Analysis

HaCaT cells were seeded into a 12-well plate (7×10⁴ cells/well), and incubated with 5 μM corylin or 20 μM neobavaisoflavone, or without drugs, for 4 hours, before subjected to UVB irradiation at 14 mJ/m² for four runs. At 24 hours, the irradiated cells were collected, washed, and fixed with 4% paraformaldehyde, before being immersed in 0.5% crystal violet solution for 15 minutes at room temperature. The cells were washed, air-dried, photographed, and immersed in 50% ethanol containing 0.1% glacial acetic acid to remove the crystal violet dye. The absorbance at 550 nm was read by a microplate reader.

### 7. Intracellular NAD⁺ Content

HaCaT cells were seeded into a 6-cm dish (4×10 5 cells/dish), and incubated with 5 μM corylin or 20 μM neobavaisoflavone, or without drugs, for 24 hours, before being harvested. The harvested cells were extracted with 250 μl of 1 M glacial formic acid saturated with butanol, incubated for 30 minutes on ice. Then, 62.5 μl of 100% TCA (w/v) was added to each sample, and the samples were incubated on ice for an additional 15 minutes. The samples were pelleted by centrifugation at 14,000×g for 5 minutes, and the supernatant was transferred to another eppendorf tube. The pellets were then washed with 125 μl of 20% TCA, and centrifuged, and the supernatant was combined with the aforementioned supernatant. The combined supernatants were used for the following tests. For analysis, each supernatant sample was divided into two samples: the test sample and the control sample, each of which was assembled with an alcohol dehydrogenase (ADH) reaction buffer (i.e., 150 μl of the supernatant, 345 μl of 360 mM Tris (pH 9.7), 240 mM lysine, 0.24% (v/v) EtOH; the control group had 5 μl of ddH₂O, and the ADH group had 5 μl of 5 mg/ml ADH). After incubation at 30° C. for 20 minutes, the absorbance of each sample was measured at 340 nm. The NAD⁺ level of the cells in each supernatant sample was determined by the test sample (the alcohol ADH catalytic group) relative to the corresponding control sample (the water group, as the basal NADH level).

### 8. Animals

All animal procedures were complied with relevant regulations and were conducted under approved protocols (Protocol #CGU15-150) by Research Guidelines for the Care and Use of Laboratory Animals, Chang Guan University. C57BL/6J male mice (34 weeks of age) were maintained at 23±1° C. and at humidity of 45-65% with a 12 h dark/light cycle. After reaching 40 weeks of age, the mice were randomly divided into two groups: Group I (HFD), fed a HFD with 54% fat (n=30), and Group II (HFD/C), fed a HFD containing corylin (1 g corylin/1 kg HFD) (or the dose of corylin was 50 mg/kg) (n=30). The composition of the test diets is summarized in Table 1.

TABLE 1
Composition of the test diets

g/kg diet

| Component | HFD | HFD/C |
|---|---|---|
| Casein | 254 | 254 |
| Cellulose | 61 | 61 |
| Sucrose | 321 | 321 |
| Soybean oil | 10 | 10 |
| Unsalted butter | 290 | 290 |
| AIN-93G Mineral mixture | 44.5 | 44.5 |
| Ain-93 Vitamin mixture | 12.5 | 12.5 |
| L-Cystine | 4 | 4 |
| Choline bitartrate | 3 | 3 |
| Corylin | — | 1 |

| | |
|---|---|
| kcal/g | 5.016 |
| CHO calorie/total calories (%) | 25.7 |
| Fat calorie/total calories (%) | 54.0 |
| Protein calorie/total calories (%) | 20.3 |

### 9. Pharmacokinetic Analysis

The blood samples of the C57BL/6J male mice (n=4) were collected at 1, 3, 6, 9, 12 and 15 h after corylin oral gavage, and the serum samples were then separated therefrom. Each serum sample (100 μl) was mixed with ice-cold acetonitrile (150 μl) at 4° C. for 1 hour, and then centrifuged at 15,000×g and 4° C. for 15 min. After centrifugation, the supernatant (150 μl) was harvested, and 150 μl ddH₂O was added. The resulting mixture was analyzed by a LC/FTMS system.

### 10. Rearing Behavior

Rearing behavior was analyzed in the C57BL/6J male mice fed a HFD with or without corylin for 35 weeks (75 weeks of age) using the OxyletPro System (HFD: n=6; HFD/C: n=9). Before the metabolic rate was monitored, the mice were individually caged for 24 h to acclimate to the system.

### 11. Rotarod Test

The C57BL/6J male mice were tested on a rotarod at 81 weeks of age. The mice were habituated for 3 days, during which they were placed on the rotarod at a constant speed (4 rpm), and had to remain on the rotarod for 1 min every day. Testing on each day consisted of five trials with a 10-minute rest between each trial. The acceleration trial began with the rotarod set at an initial rotating speed of 4 rpm, and accelerated to a maximum of 40 rpm within 5 minutes (HFD: n=7; HFD/C: n=5). The constant trial began with the rotarod set at a rotating speed of 4 rpm, and lasted for a maximum of 200 seconds (HFD: n=10; HFD/C: n=8). The latency to falling was recorded, and the average latency to falling was calculated for each trial.

### 12. Serum Parameter Analysis

To determine the fasting blood glucose level, the C57BL/6J male mice at 102 weeks of age (n=9) were stayed fasted for 16 hours. The blood samples were obtained by cutting the tip of tail, and the blood glucose was measured by ACCU-CHEK (Roche).

The C57BL/6J male mice were sacrificed, and their whole bloods for serum biochemistry were collected before sacrifice (102 weeks of age) (n=5). The level of serum LDL, TG, and total cholesterol was measured with specific reagent kits (Fortress Diagnostics). The level of serum glucose and high-density lipoprotein (HDL) was measured with specific reagent kits (Randox). The level of serum AST and creatinine was measured using a Fuji Dri-Chem 4000i analyzer (Fujifilm, Tokyo, Japan).

### 13. Statistics and Reproducibility

Graphic visualization and statistical analyses were performed by GraphPad Prism 6. All values are exhibited as the mean±S.D., and the exact p value was provided in figures or legend. For the experiments of the western blotting, the SA-β-gal staining, the rearing behavior, and the rotarod test, significance was determined via Student's t-test. For the PDL of the HUVECs, significance was determined by two-way ANOVA (multiple comparisons). At least three independent replicates were performed for each experiment. For the survival curve of the mouse model, significance was determined by the Gehan-Breslow-Wilcoxon test.

### Example 1

### Isolation and Identification of Corylin and Neobavaisoflavone

The corylin and neobavaisoflavone were extracted and purified from Psoralea corylifolia in accordance with the procedures described in the section of “Materials and Methods.” The identities of the purified compounds (i.e., corylin and neobavaisoflavone) were independently confirmed by NMR proton spectroscopy.

The NMR proton spectra of corylin were as follows: corylin: white power; formula: C₂₀H₁₆O₄; MP: 245-255° C.; UV λₘₐₓ(MeOH) nm: 305, 247; IR νₘₐₓ(KBr) cm⁻¹: 3235, 2927, 1625, 1495, 1375, 1274, 1184 ESI-MS m/z: 321 [M+1]⁺, 343 [M+Na]⁺, 359 [M+K]⁺; ¹H-NMR (acetone-d₆, 400 MHz) δ_{H}: 9.62 (1H, s, OH-7), 8.19 (1H, s, H-2), 8.06 (1H, d, J=8.8 Hz, H-5), 7.38 (1H, dd, J=8.0, 2.0 Hz, H-6′), 7.32 (1H, s, H-2′), 7.01 (1H, dd, J=8.8, 2.0 Hz, H-6), 6.91 (1H, d, J=2.0 Hz, H-8), 6.77 (1H, d, J=8.0 Hz, H-5′), 6.44 (1H, d, J=10.0 Hz, H-1″), 5.77 (1H, d, J=10.0 Hz, H-2″), 1.43 (6H, s, CH₃, H-4″,5″); ¹³C-NMR (acetone-d₆, 100 MHz) δ_{C}: 175.1 (C═O), 162.7 (C-7), 158.2 (C-9), 153.2 (C-4′), 152.9 (C-2), 131.3 (C-3″), 130.1 (C-6′), 128.0 (C-5), 127.5 (C-2′), 125.3 (C-1′), 124.4 (C-3), 122.4 (C-4″), 121.3 (C-3′), 118.1 (C-10), 116.1 (C-5′), 115.1 (C-6), 102.7 (C-8), 76.6 (C-2″), 27.8 (C-5″, 6″).

The NMR proton spectra of neobavaisoflavone were as follows: neobavaisoflavone: white power; formula: C₂₀H₁₈O₄; MP. 180-195° C.; UV λₘₐₓ(MeOH) nm: 308, 259, 249; IR νₘₐₓ(KBr) cm⁻¹: 3362, 3125, 2968, 2923, 1625, 1381, 1092, 858 ESI-MS m/z: 323 [M+1]⁺; ¹H NMR (DMSO-d₆, 400 MHz) δ_{H}: 9.58 (1H, br.s. OH), 8.35 (1H, br.s, OH), 8.12 (1H, s, H-2), 8.08 (1H, d, J=8.0 Hz, H-5), 7.38 (1H, d, J=2.0 Hz, H-1′), 7.30 (1H, dd, J=8.0, 2.0 Hz, H-4′), 7.00 (1H, dd, J=8.8, 2.0 Hz, H-6), 6.91 (1H, d, J=2.0 Hz, H-8), 6.88 (1H, d, J=8.0 Hz, H-5′), 5.40 (1H, t, J=7.2 Hz, H-2″), 3.38 (2H, d, J=7.2 Hz, H-1″), 1.75 (3H, s, CH₃), 1.73 (3H, s, CH₃).

### Example 2

### Effects of Corylin and/or Neobavaisoflavone on Ameliorating Cellular Senescence

### 2.1 Population Doubling Level (PDL)

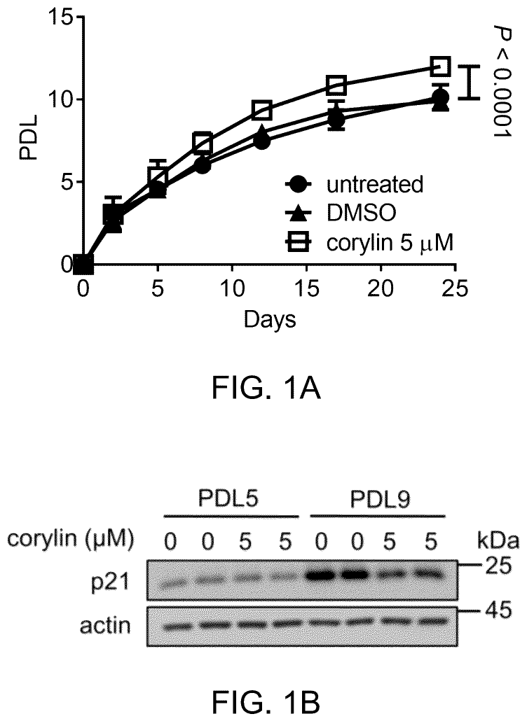

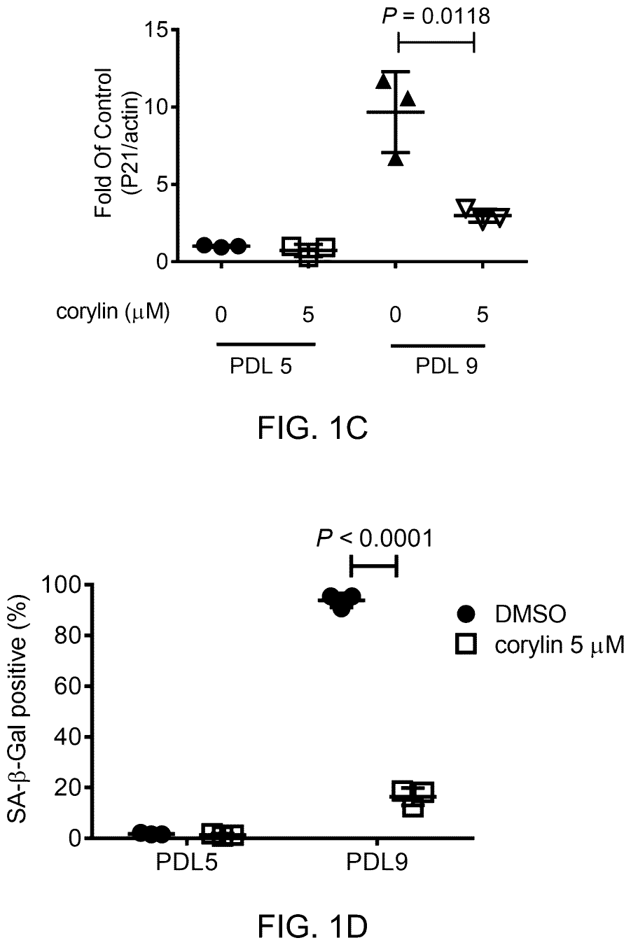

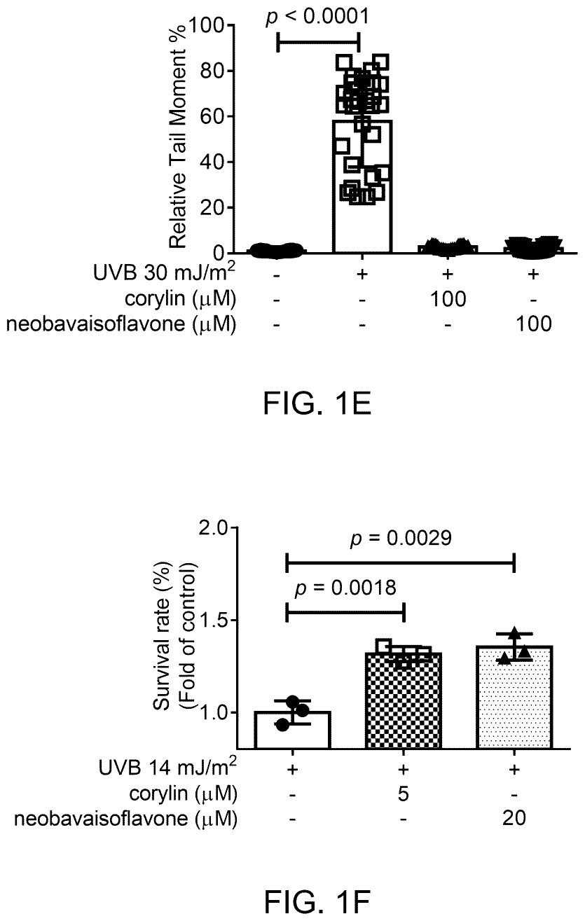

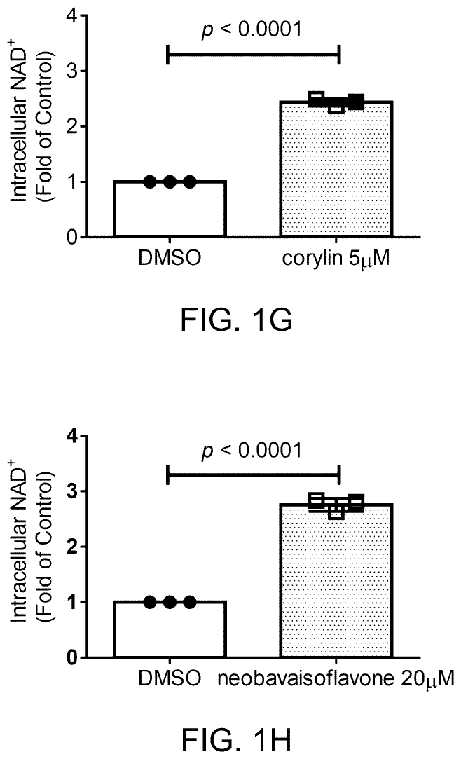

The effects of the corylin and/or the neobavaisoflavone of Example 1 on mammalian cellular senescence was investigated by monitoring the population doubling level (PDL). It was found that corylin could increase the PDL of HUVECs (FIG. 1A).

Further, it has been reported that p21 and SA-β-gal are representatives of replicative exhaustion signature in mammalian cells, thus the effect of corylin and/or neobavaisoflavone on the expression of p21 and SA-β-gal were explored. References are made to FIGS. 1B-1C, which indicated that in the late stage of HUVECs (i.e., HUVECs at PDL9), but not in the early-middle stage of HUVECs (i.e., HUVECs at PDL5), the expression level of p21 increased in the absence of corylin, suggesting that the cells were in the status of cell cycle arrest, whereas the expression level of p21 decreased in the presence of corylin. Further, corylin also resulted in a decrease in the number of the SA-β-gal-positive senescent cells in the late stage of HUVECs (i.e., HUVECs at PDL9), as compared with that in the control group without corylin (FIG. 1D). Taken together, these data revealed that corylin could ameliorate cellular senescence in mammalian cells.

### 2.2 DNA Breakage and Cellular Apoptosis

In addition to natural aging, there are some common factors in life, such as UV irradiation, etc., that may incur cellular senescence, and thus in this example, whether corylin and the neobavaisoflavone of Example 1 could prevent the cellular senescence induced by UVB were investigated by monitoring the levels of DNA breakage and cellular apoptosis. Results are provided in FIGS. 1E-1F.

It was found that both corylin and neobavaisoflavone could completely suppressed the UVB-induced DNA breakage (FIG. 1E), indicating that pretreatment of corylin or neobavaisoflavone could effectively prevent the cells from UVB-induced DNA breakage. Also, the results in FIG. 1F evidently showcased that corylin or neobavaisoflavone significantly increased the survival rate of the cell under the stress of UVB irradiation, indicating that pretreatment of corylin or neobavaisoflavone on cells may effectively reduce cell apoptosis caused by UVB irradiation.

### 2.3 Intracellular NAD⁺ Level

Several studies have shown that increase in the level of NAD⁺ helps ameliorate aging-associated diseases. Armed with such knowledge, the present example investigated the effect of corylin or neobavaisoflavone on the NAD⁺ level in cells. Results are provided in FIGS. 1G-1H.

It was found that both corylin and neobavaisoflavone could independently elevate the NAD⁺ level in the cells, demonstrating that both corylin and neobavaisoflavone could mitigate the level of senescence in the cells.

### Example 3

### Corylin Delayed Aging Process in Mice

### 3.1 Survival Rate of the Aged Mice

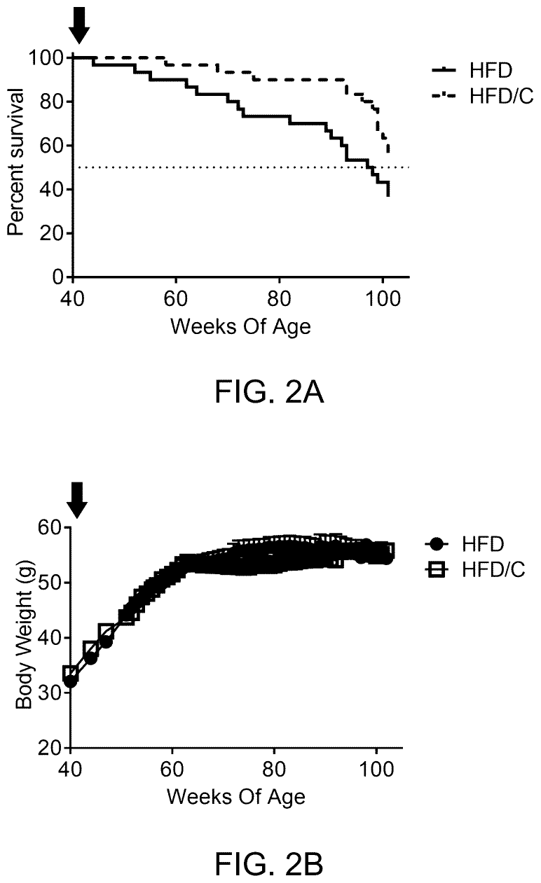

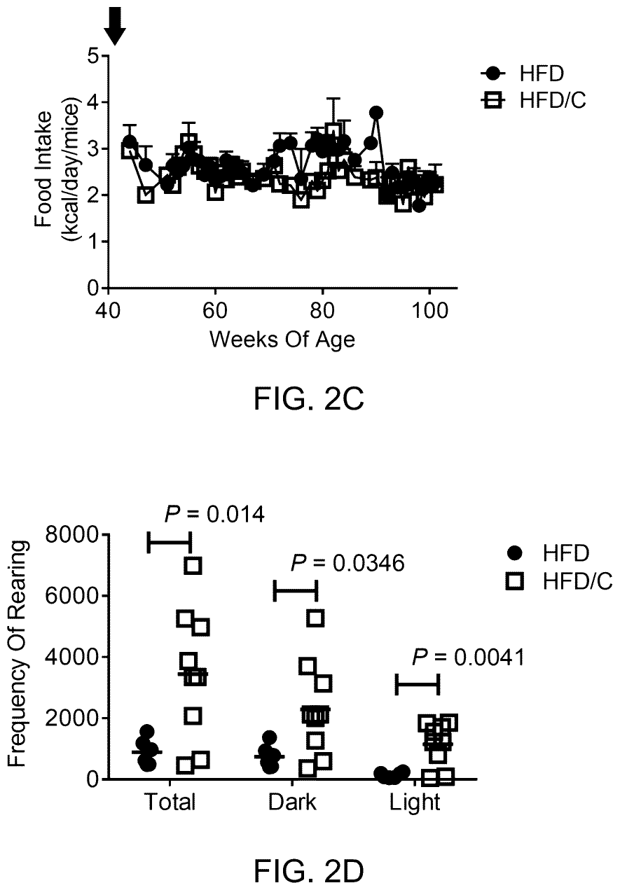

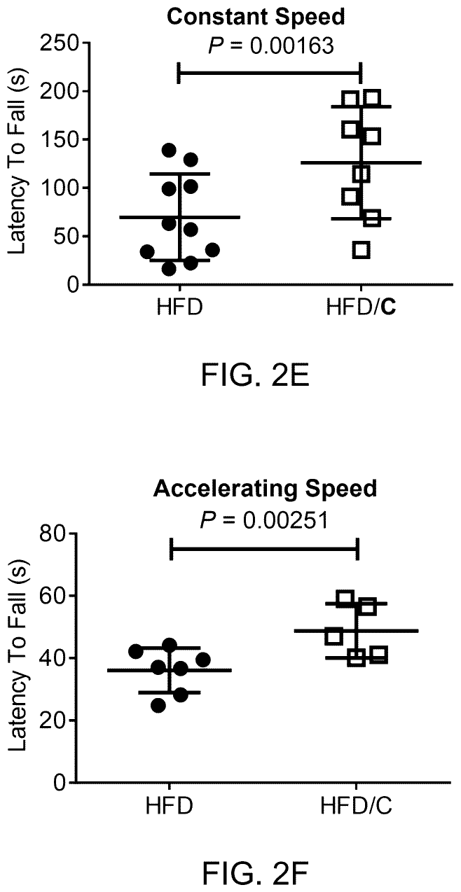

The ability of corylin to delay the aging process in mice was examined in the present example. To this purpose, aged C57BL/6J mice (forty-week-old) were fed with a HFD or a HFD plus 0.1% (w/w) corylin (HFD/C) ad libitum for the rest of their lives. Results are shown in FIGS. 2A to 2C.

As depicted in FIG. 2A, the survival curves of the mice in the HFD and the HFD/C groups started to diverge after four weeks (i.e., at 44 weeks of age) and the divergent continued till the end of experiments. The result confirmed that the mice in the HFD/C group overall exhibited higher survival rate as compared with that of the mice in the HFD group. By 102 weeks of age, 63.3% of the mice in the HFD-fed control group had died, whereas only 43.3% of the mice in the HFD/C-fed test group had died. The results evidenced that corylin supplement significantly increased the lifespan of aged mice fed on high fat diet. Note that during the entire course of the experiment, the trajectories of the body weight and the food intake did not diverge between the two groups (FIGS. 2B-2C), suggesting that the benefits on increasing lifespan was exerted by corylin instead of by reduced caloric intake. The pharmacokinetics of corylin in mice was closely monitored via tracking the circulating level of corylin after oral gavage. The results of the average serum level of corylin along the course are summarized in Table 2, in which the average serum level of corylin reached 0.68 μM at 1 hour after corylin oral gavage and remained at 0.26 μM at 15 hour. In accordance with the forgoing results, the daily dose of corylin was calculated to be 50 mg per kg body weight based on the food intake of mice, which was approximately 2 g of HFD plus corylin (0.1% w/w) per day in mice with an average body weight of 40 g.

These data suggested that corylin supplement could delay the aging process which leads to prolonging longevity in aged mice, even under metabolic stress.

TABLE 2
The average serum level of corylin along the course

| | Time after oral gavage (hour) | | | | | |
|---|---|---|---|---|---|---|
| | 1 | 3 | 6 | 9 | 12 | 15 |
| Serum | 0.68 ± | 0.50 ± | 0.49 ± | 0.54 ± | 0.39 ± | 0.26 ± |
| level | 0.05 | 0.17 | 0.14 | 0.26 | 0.18 | 0.13 |
| (μM) | | | | | | |

### 3.2 Physical Changes of the Aged Mice

The beneficial effects of corylin on age-associated physical dysregulations were validated in the present example. The beneficial effects in respect of age-related physical dysfunctions in aged HFD-fed mice were addressed by the observation of the rearing behavior (including vertical activity and behavior), and the rotarod tests (including rotarod at constant speed or at accelerating speed). Results are illustrated in FIGS. 2D to 2F.

It was found that the HFD/C-fed mice exhibited enhanced activity as compared with that of the HFD-fed mice (FIG. 2D). The rotarod test, which revealed physical function of muscle strength and balance of the test animal, was utilized to examine the motor coordination of the aged mice. According to FIGS. 2E and 2F, the HFD/C-fed mice exhibited better motor skills (i.e., having longer latency to falling) than the HFD-fed mice, either in rotarod at constant speed or at accelerating speed.

Taken together, the results indicated that corylin may ameliorate the age-related decline in terms of physical functions.

### 3.3 Metabolic Alteration of the Aged Mice

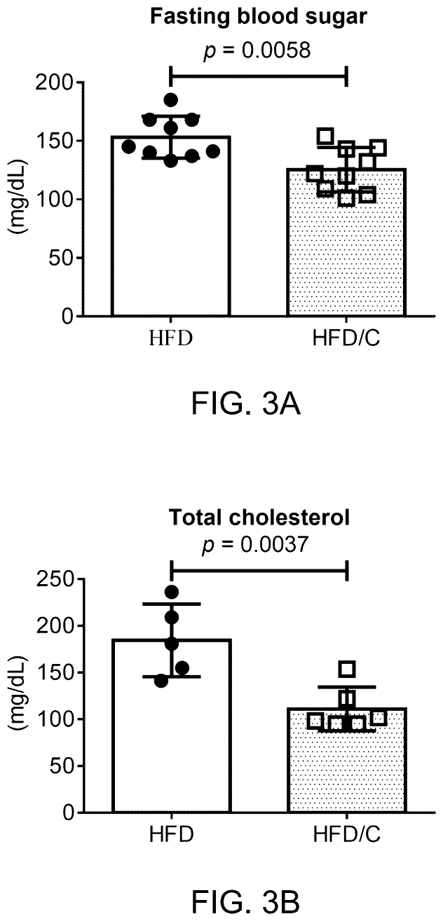

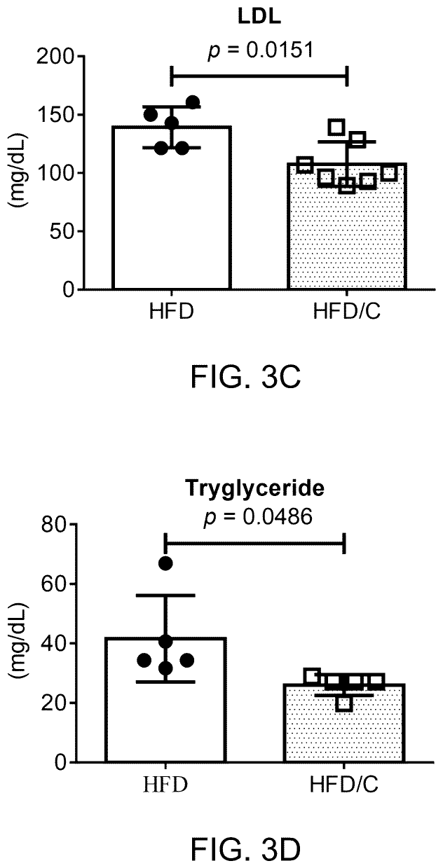

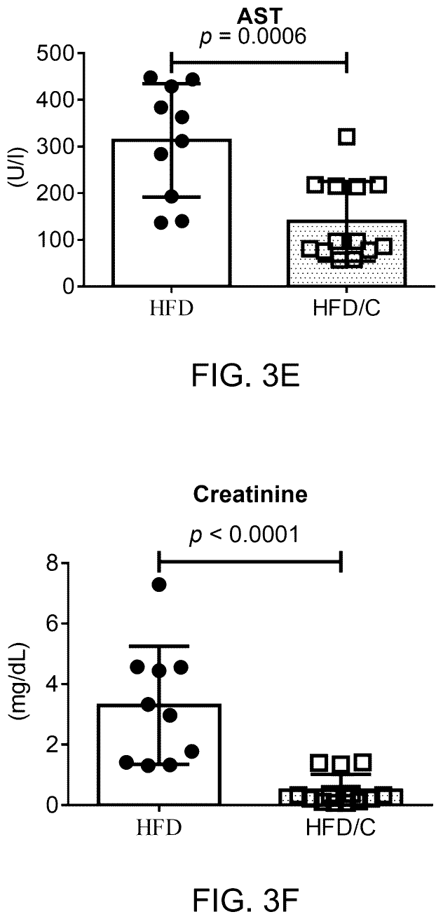

The metabolic alteration of the aged mice was examined in this example by monitoring several metabolic parameters that include fasting blood glucose, total cholesterol, LDL, and TG levels, which reflect the risk of aging-associated pathology during aging. To this purpose, the blood of each aged male mice in HFD/C and HFD groups (102 weeks old) was collected and analyzed for fasting serum parameters. Results are provided in FIGS. 3A-3F and in Table 3.

For serum lipid parameters, the levels of the fasting blood glucose, the total cholesterol, the LDL, and the TG was decreased in the HFD/C-fed mice, as compared with those in the HFD-fed mice (FIGS. 3A-3D, Table 3). These results confirmed that corylin could combat aging via improving serum lipid parameters, which in turn leads to improved overall health of aged mice.

TABLE 3
The serum parameters in the HFD- and the HFD/C-fed mice

| | HFD | HFD/C | p value |
|---|---|---|---|
| Fasting blood glucose (mg/dl) | 153.1 ± 5.9 | 125.4 ± 6.3 | 0.0058 |
| Total cholesterol (mg/dl) | 184.4 ± 17.37 | 110.9 ± 9.50 | 0.0037 |
| LDL (mg/dl) | 139.3 ± 7.82 | 107.7 ± 7.2 | 0.0151 |
| HDL (mg/dl) | 100.6 ± 8.25 | 92.7 ± 9.9 | >0.05 |
| TG (mg/dl) | 41.63 ± 6.5 | 26.06 ± 1.57 | 0.0486 |
| AST (U/I) | 313.4 ± 38.4 | 139.7 ± 23.7 | 0.0006 |
| Creatinine (mg/dl) | 3.3 ± 0.6 | 0.5 ± 0.1 | 0.0001 |

Further, markers with respect of hepatic and renal functions: AST (a hepatic damage marker) and creatinine (a marker of renal function) were also evaluated, and the results are provided in FIGS. 3E-3F and in Table 3.

A lower level of AST and creatinine in the HFD/C-fed aged mice as compared to that of the HFD-fed aged mice was noted, indicating that corylin also helped delay aging via preventing aging-associated organ functional decline.

In sum, the data disclosed herein demonstrated that the present corylin and neobavaisoflavone independently possessed anti-aging properties reflected in many aspects, and thus the corylin or the neobavaisoflavone may be used as a potential therapeutic agent for treating symptoms or diseases associated with senescence.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method for increasing muscle strength or motor coordination; reducing muscle weakness, loss of balance, or skin wrinkle, comprising administering to the subject an effective amount of corylin and/or neobavaisoflavone.

2. (canceled)

3. The method of claim 1, wherein the corylin and neobavaisoflavone are respectively administered in the amount of about 1-10 mg/kg and about 1-10 mg/kg.

4. The method of claim 1, wherein the subject is a human.
